(19) United States
(12) Patent Application Publication
(10) Pub. No.: US 2016/0171724 A1
(43) Pub. Date: Jun. 16, 2016

(54) METHODS AND SYSTEMS FOR REAL-TIME IMAGE RECONSTRUCTION WITH ARBITRARY TEMPORAL WINDOWS

(71) Applicant: General Electric Company, Schenectady, NY (US)

(72) Inventors: Brian Edward Nett, Brookfield, WI (US); Jed Douglas Pack, Glenville, NY (US); Michael Richard Jones, Milwaukee, WI (US)

(21) Appl. No.: 14/571,119

(22) Filed: Dec. 15, 2014

(51) Int. Cl. G06T 11/00; G06T 11/60; G06T 15/08; A61B 6/03; A61B 6/00

(52) U.S. Cl. CPC G06T 11/003; A61B 6/032; A61B 6/463; G06T 15/08; G06T 11/60; G06T 2207/10081; G06T 2207/30048; G06T 2207/30196; G06T 2210/41; G06T 2211/428

(57) ABSTRACT

Various methods and systems for generating a computed tomography image in real-time are provided. In one embodiment, a method for imaging comprises acquiring x-ray projection data; calibrating the x-ray projection data; generating at least two basis image volumes by reconstructing the calibrated x-ray projection data, each of the at least two basis image volumes including different centerviews and a temporal width corresponding to an x-ray source rotation between 180 degrees and 360 degrees; and generating a final image volume by Fourier blending the at least two basis image volumes based on a selected temporal window width. In this way, a user may scroll between Fourier-blended images of varying durations of temporal window in real-time to select an optimal image for review without a reconstruction of calibrated x-ray projection data for each image.

### FIELD

Embodiments of the subject matter disclosed herein relate to non-invasive diagnostic imaging, and more particularly, to real-time image reconstruction with an arbitrary temporal window.

### BACKGROUND

Non-invasive imaging technologies allow images of the internal structures of a patient or object to be obtained without performing an invasive procedure on the patient or object. In particular, technologies such as computed tomography (CT) use various physical principles, such as the differential transmission of x-rays through the target volume, to acquire image data and to construct tomographic images (e.g., three-dimensional representations of the interior of the human body or of other imaged structures).

In CT image reconstruction, there is a trade-off between high temporal resolution and high signal-to-noise ratio. For example, when imaging anatomy that has significant motion such as the heart, high temporal resolution is preferable to avoid blurring in the image. On the other hand, when imaging anatomy that has little motion such as the abdomen, high signal-to-noise ratio is preferable to reduce noise and improve lesion detectability. The choice of temporal window used for image reconstruction, defined by the number of views (e.g., the duration of acquired data) used to reconstruct an image, affects temporal resolution and signal-to-noise ratio. In modern CT scanners, there are two typical choices of temporal window when reconstructing an image: half scan and full scan. A half-scan reconstruction uses a minimal number of data views (e.g., 180 degrees plus a scanner fan angle) to reconstruct the image, while a full-scan reconstruction uses 360 degrees of data views. This choice must be made once per reconstruction and there is a wait time associated with each reconstruction.

### BRIEF DESCRIPTION

In one embodiment, a method for imaging comprises acquiring x-ray projection data; calibrating the x-ray projection data; generating at least two basis image volumes by reconstructing the calibrated x-ray projection data, each of the at least two basis image volumes including different centerviews and a temporal width corresponding to an x-ray source rotation between 180 degrees and 360 degrees; and generating a final image volume by Fourier blending the at least two basis image volumes based on a selected temporal window width. In this way, a user may scroll between Fourier-blended images of varying durations of temporal window in real-time to select an optimal image for review without a reconstruction of calibrated x-ray projection data for each image.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a pictorial view of an imaging system according to an embodiment of the invention.

FIG. 2 is a block schematic diagram of an exemplary imaging system according to an embodiment of the invention.

FIG. 3 is a high-level flow chart illustrating an example method for reconstructing an image volume with a user-specified temporal window according to an embodiment of the invention.

FIG. 4 is a high-level block diagram illustrating an example method of reconstructing an image volume with a user-specified temporal window from two half-scan reconstructed image volumes according to an embodiment of the invention.

FIG. 5 is a high-level flow chart illustrating an example method for generating a weighting function according to an embodiment of the invention.

FIG. 6 shows a set of graphs illustrating an example construction of a weighting function according to an embodiment of the invention.

FIG. 7 shows a pictorial overview of generating an image volume according to an embodiment of the invention.

FIG. 8 shows a set of example image volumes with different temporal windows according to an embodiment of the invention.

### DETAILED DESCRIPTION

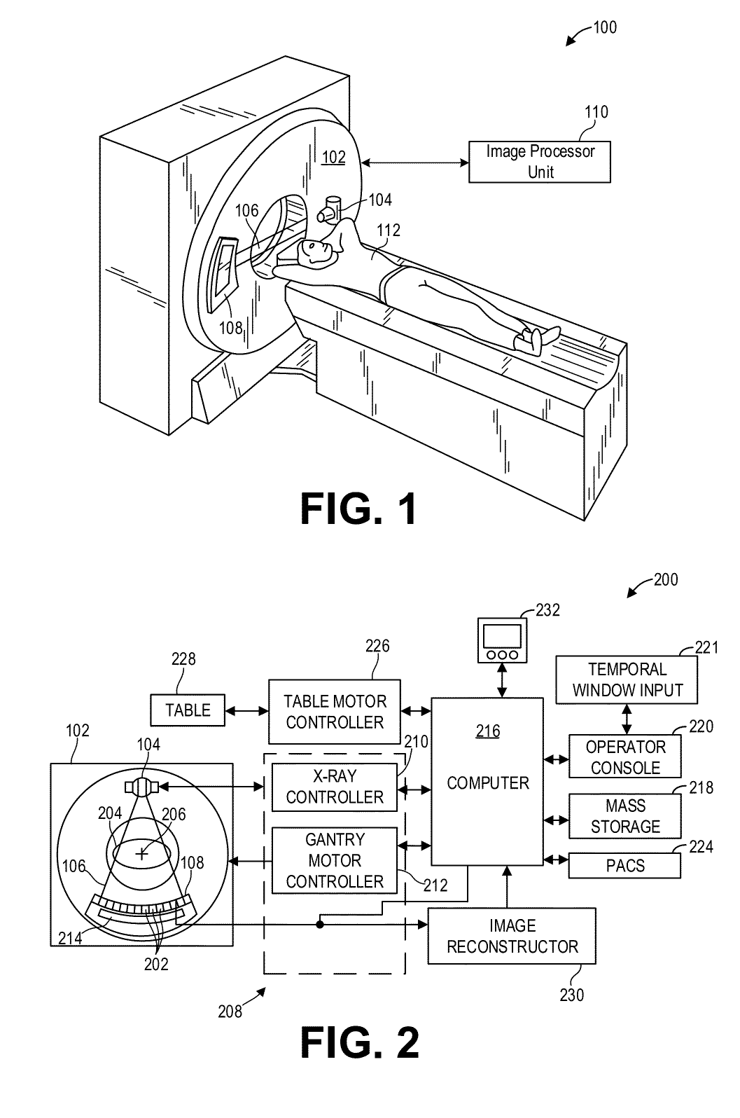

The following description relates to various embodiments of medical imaging systems. In particular, methods and systems are provided for real-time generation of an image with a selected temporal reconstruction window. An example of a computed tomography (CT) imaging system that may be used to acquire images processed in accordance with the present techniques is provided in FIGS. 1 and 2. Though a CT system is described by way of example, it should be understood that the present techniques may also be useful when applied to images acquired using other imaging modalities, such as tomosynthesis, MRI, C-arm angiography, and so forth. The present discussion of a CT imaging modality is provided merely as an example of one suitable imaging modality.

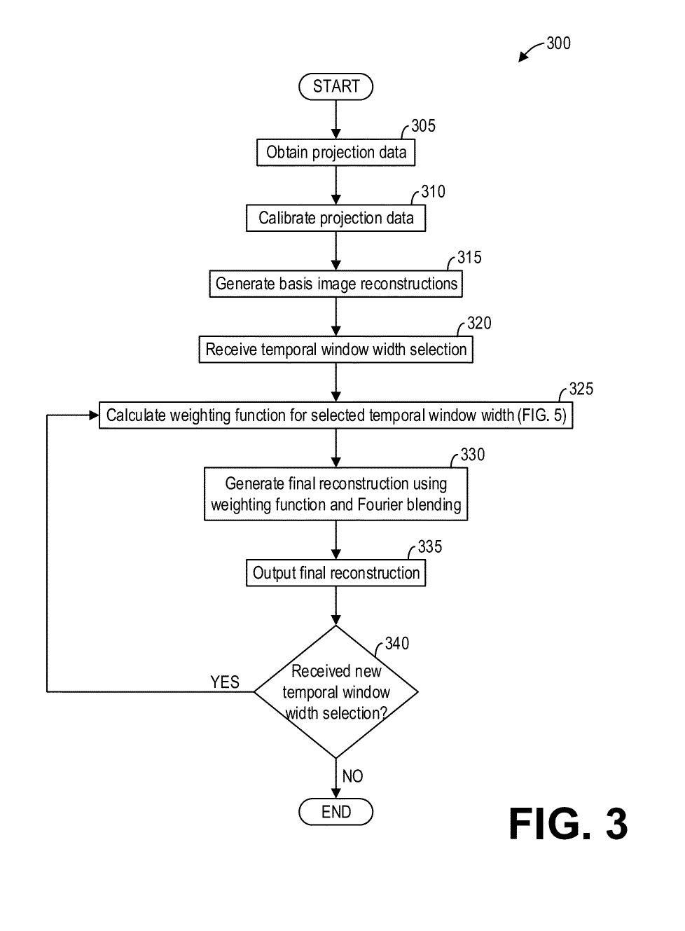

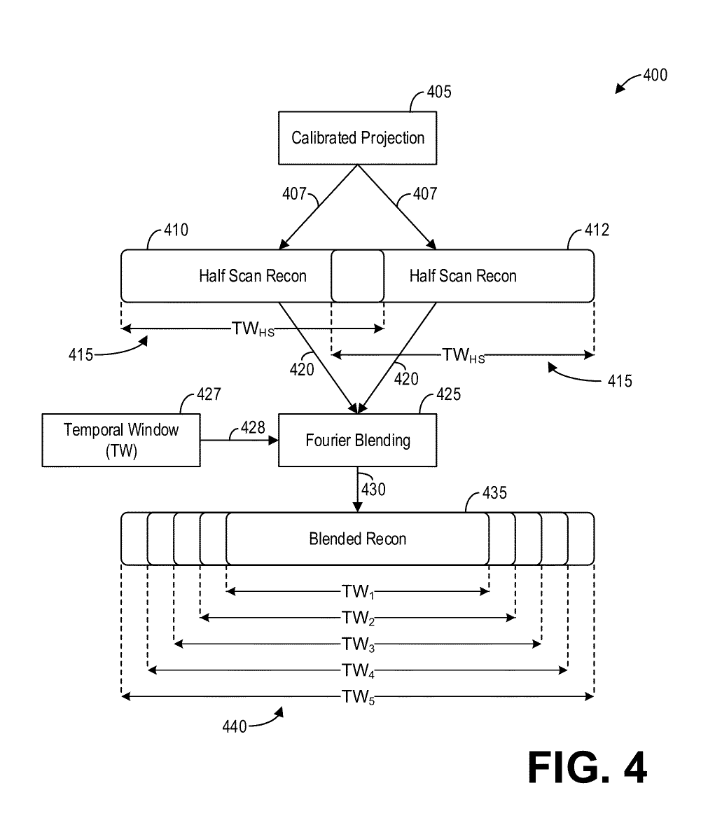

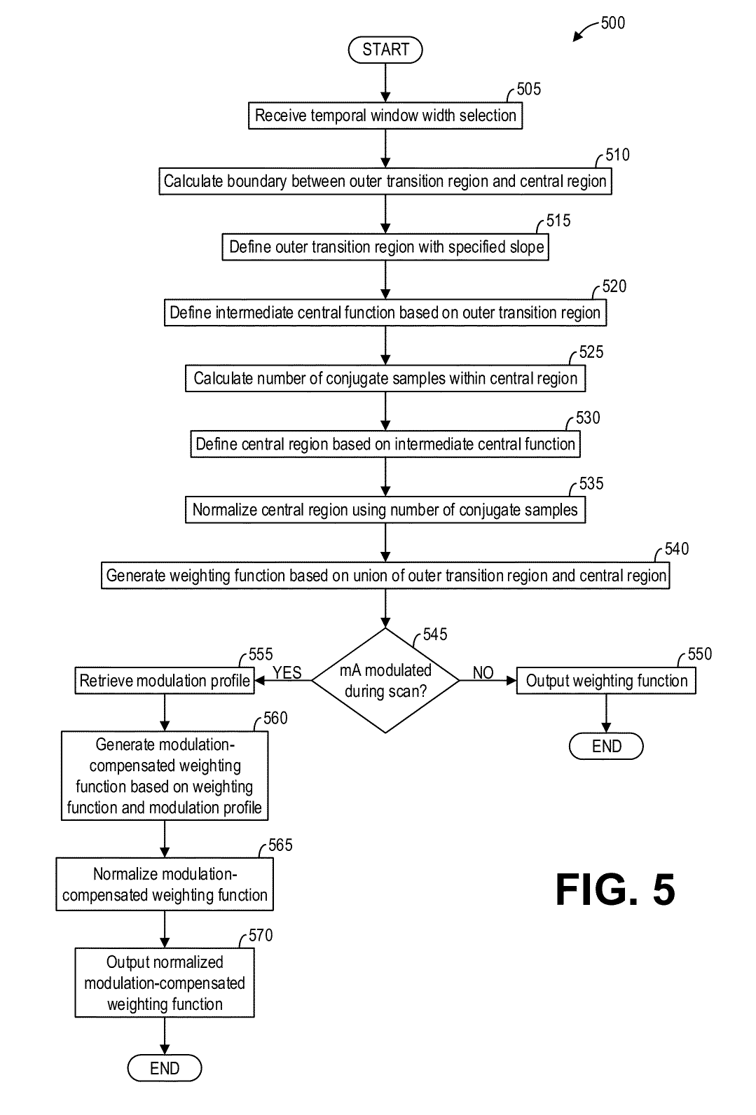

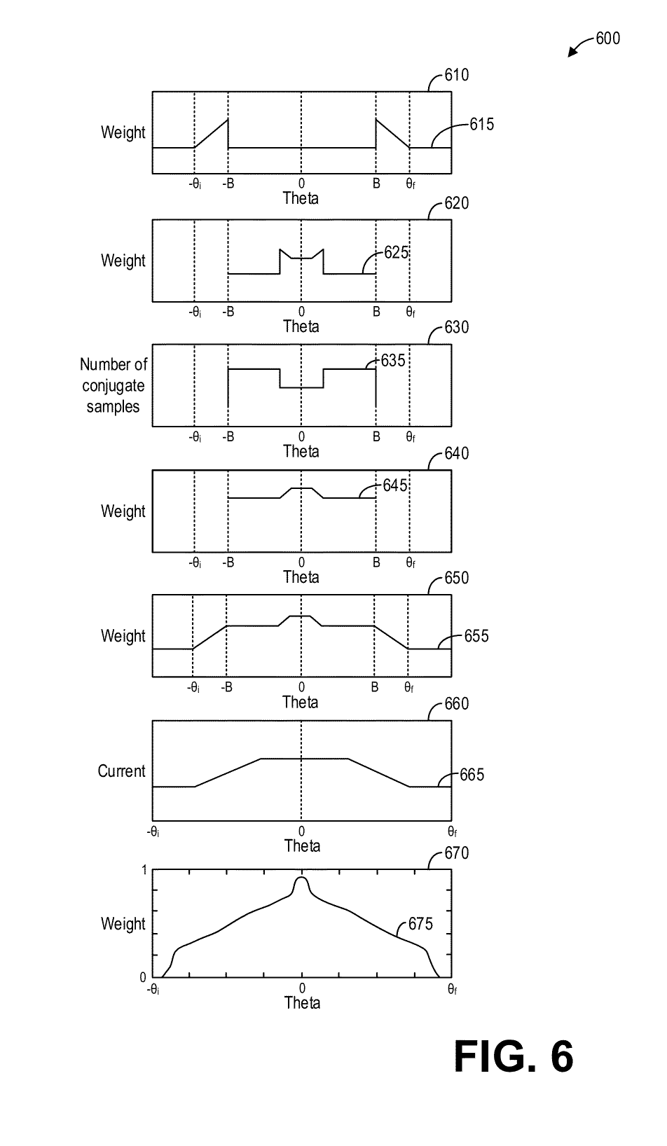

For a CT imaging system such as shown in FIGS. 1 and 2, there is often built-in data padding during data acquisition. For example, in cardiac CT, acquiring data over a range of phases of the cardiac cycle enables flexibility in reconstructing an image of the heart at the best cardiac phase, which is not known a priori. It is only during image reconstruction that a minimal amount of data centered on the desired cardiac phase is used (e.g., half-scan). In body scans, at least a full-scan of data is acquired with wide-cone scanners for proper volumetric reconstruction. In both of these cases, a greater than minimal amount of data is acquired to reconstruct an image. Therefore, most scans acquire sufficient data to reconstruct an image using a minimal amount of data for high temporal resolution, as well as additional data for an image with a higher signal-to-noise ratio (SNR). For scans that acquire data from more than one full (360 degree) rotation of the gantry, there is an additional opportunity to utilize even more data. Thus, FIG. 3 illustrates a method for the real-time generation of an image with a selected temporal window. The method comprises Fourier blending two or more reconstructed basis images, and thus enables a user the flexibility to scroll in real-time between images reconstructed using varying durations of temporal window as depicted in FIG. 4. The basis images may be Fourier blended according to a weighting function to generate an image with a selected temporal window. A method for constructing such a weighting function is depicted in FIGS. 5 and 6.

While cardiac imaging with a CT imaging system is utilized as a non-limiting illustrative example throughout the present disclosure, a person of ordinary skill in the art having the benefit of this disclosure will readily appreciate that the novel features of the present technology can be extended to a wide variety of imaging systems and applied to a wide variety of subjects.

FIG. 1 illustrates an exemplary CT system 100 configured to allow fast and iterative image reconstruction. Particularly, the CT system 100 is configured to image a subject such as a patient, an inanimate object, one or more manufactured parts, and/or foreign objects such as dental implants, stents, and/or contrast agents present within the body. In one embodiment, the CT system 100 includes a gantry 102, which in turn, may further include at least one x-ray radiation source 104 configured to project a beam of x-ray radiation 106 for use in imaging the patient. Specifically, the radiation source 104 is configured to project the x-rays 106 towards a detector array 108 positioned on the opposite side of the gantry 102. Although FIG. 1 depicts only a single radiation source 104, in certain embodiments, multiple radiation sources may be employed to project a plurality of x-rays 106 for acquiring projection data corresponding to the patient at different energy levels.

In certain embodiments, the CT system 100 further includes an image processing unit 110 configured to reconstruct images of a target volume of the patient using an iterative or analytic image reconstruction method. For example, the image processing unit 110 may use an analytic image reconstruction approach such as filtered back projection (FBP) to reconstruct images of a target volume of the patient. As another example, the image processing unit 110 may use an iterative image reconstruction approach such as conjugate gradient (CG), maximum likelihood expectation maximization (MLEM), or model-based iterative reconstruction (MBIR) to reconstruct images of a target volume of the patient.

FIG. 2 illustrates an exemplary imaging system 200 similar to the CT system 100 of FIG. 1. In accordance with aspects of the present disclosure, the system 200 is configured to reconstruct images with a user-specified temporal window in real-time. In one embodiment, the system 200 includes the detector array 108 (see FIG. 1). The detector array 108 further includes a plurality of detector elements 202 that together sense the x-ray beams 106 (see FIG. 1) that pass through a subject 204 such as a patient to acquire corresponding projection data. Accordingly, in one embodiment, the detector array 108 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

In one embodiment, the system 200 includes a control mechanism 208 to control movement of the components such as rotation of the gantry 102 and the operation of the x-ray radiation source 104. In certain embodiments, the control mechanism 208 further includes an x-ray controller 210 configured to provide power and timing signals to the radiation source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The data sampled and digitized by the DAS 214 is transmitted to a computing device 216. In one example, the computing device 216 stores the data in a storage device 218. The storage device 218, for example, may include a hard disk drive, a floppy disk drive, a compact disk-read/write (CD-R/W) drive, a Digital Versatile Disc (DVD) drive, a flash drive, and/or a solid-state storage device.

Additionally, the computing device 216 provides commands and parameters to one or more of the DAS 214, the x-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

The operator console 220 may be operably connected to a temporal window input 221 to allow the operator to specify a temporal window width of a displayed image. The temporal window input 221 may comprise any suitable user input device, for example a slider, a potentiometer, and so on. While temporal window input 221 is depicted as an independent block, in some examples operator console 220 may comprise temporal window input 221. Temporal window input 221 combined with the methods described further herein enable the operator to scroll in real-time between images reconstructed using varying durations of temporal window.

Although FIG. 2 illustrates only one operator console 220, more than one operator console may be coupled to the system 200, for example, for inputting or outputting system parameters, requesting examinations, and/or viewing images. Further, in certain embodiments, the system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks.

In one embodiment, for example, the system 200 either includes, or is coupled to a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a motorized table 228. Particularly, the table motor controller 226 moves the table 228 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized x-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the system 200 and instead the computing device 216 may perform one or more functions of the image reconstructor 230. Moreover, the image reconstructor 230 may be located locally or remotely, and may be operatively connected to the system 100 using a wired or wireless network. Particularly, one exemplary embodiment may use computing resources in a “cloud” network cluster for the image reconstructor 230.

In one embodiment, the image reconstructor 230 stores the images reconstructed in the storage device 218. Alternatively, the image reconstructor 230 transmits the reconstructed images to the computing device 216 for generating useful patient information for diagnosis and evaluation. In certain embodiments, the computing device 216 transmits the reconstructed images and/or the patient information to a display 232 communicatively coupled to the computing device 216 and/or the image reconstructor 230.

The various methods and processes described further herein may be stored as executable instructions in non-transitory memory on a computing device in system 200. In one embodiment, image reconstructor 230 may include such instructions in non-transitory memory, and may apply the methods after reconstructing an image from scanning data. In another embodiment, computing device 216 may include the instructions in non-transitory memory, and may apply the methods to a reconstructed image after receiving the reconstructed image from image reconstructor 230. In yet another embodiment, the methods and processes described herein may be distributed across image reconstructor 230 and computing device 216.

In one embodiment, the display 232 allows the operator to evaluate the imaged anatomy. The display 232 may also allow the operator to select a volume of interest (VOI) and/or request patient information, for example, via graphical user interface (GUI) for a subsequent scan or processing.

FIG. 3 is a high-level flow chart illustrating an example method 300 for reconstructing an image volume with a user-specified temporal window according to an embodiment of the invention. In particular, method 300 relates to the reconstruction of intermediate basis images and the real-time generation of an image with a specified temporal window from the basis images. Method 300 may be carried out using the system and components depicted in FIGS. 1 and 2, however the method may be applied to other systems and components without departing from the scope of the present disclosure.

Furthermore, while cardiac imaging is utilized as a non-limiting illustrative example throughout the present disclosure, method 300 may also be utilized for real-time image reconstructions for other organ anatomical imaging, such as lung imaging. Furthermore, while organ anatomical imaging is utilized as a non-limiting illustrative example throughout the present disclosure, a person of ordinary skill in the art having the benefit of this disclosure will readily appreciate that method 300 may also be utilized for real-time reconstruction of an image with a specified temporal window by any other imaging system configured to image any structure or structures in general.

Method 300 may begin at 305. At 305, method 300 may include obtaining x-ray projection data. X-ray projection data may be acquired over at least a full rotation (360°) of the gantry 102. At 310, method 300 may include calibrating the projection data. Calibrating the projection data may include, but is not limited to, applying gain calibrations and adjustments to the projection data to prepare the projection data for image reconstruction.

At 315, method 300 may include generating basis image reconstructions, or simply basis images, from the calibrated projection data. The basis images may be generated from the calibrated projection data using any suitable image reconstruction technique, including but not limited to an analytic reconstruction framework such as FBP, an iterative reconstruction framework such as MBIR or CG, and so on. In one example, each basis image may be reconstructed from projection data over a view range of 180 degrees plus the system fan angle. In other words, each basis image may comprise a standard half-scan, or short-scan, image reconstruction. The basis images may span the full breadth of projection data. That is, the union of the two view ranges may be the entire 360 degree view range. Furthermore, each basis image may have a different centerview, that is, each basis image may correspond to a different time point during the scan. While half-scan basis image reconstructions are described herein, such basis images are exemplary. It should be appreciated that in some examples, each basis image may be reconstructed from projection data acquired over a view range larger than 180 degrees. For example, each basis image may have a temporal window corresponding to an x-ray source rotation between 180 degrees and 360 degrees.

In one example, the basis images may have a non-zero overlap in their respective projection data. For example, a maximum separation in centerview angle of the basis images may be defined as 180 degrees minus a specified transition parameter λₜᵣₐₙₛ and a feathering parameter λ_{f}. Such overlap parameters may be preset or may be adjustable by an operator via operator console 220. As an illustrative example, the overlap parameters may be preset such that the transition parameter λₜᵣₐₙₛ equals 36 degrees and the feathering parameter λ_{f} equals 18 degrees. In this example, the maximum separation of centerview angles between the basis images may equal 126 degrees. In other examples, the transition parameter λₜᵣₐₙₛ may be greater than or less than 36 degrees and the feathering parameter λ_{f} may be greater than or less than 18 degrees, so that the maximum separation of centerview angles may be greater than or less than 126 degrees.

Continuing at 320, method 300 may include receiving a temporal window width selection. For example, the temporal window width may be selected by a user via temporal window width input 221. In one example, the temporal window width selection received may initially comprise a default setting of a half-scan temporal window. In another example, the temporal window width selection may be initially set to any width specified by a user. In yet another example, the temporal window width may be automatically selected to optimize one or more image parameters. For example, the temporal window width may be automatically selected to optimize a noise level in the final image.

At 325, method 300 may include calculating a weighting function for the selected temporal window width. In one example, calculating a weighting function may simply comprise obtaining the weighting function from a look-up table, where the look-up table comprises a pre-calculated weighting function for each possible temporal window width. As another example, the weighting function may be calculated in real-time in order to account for a given system configuration, which may include, as non-limiting examples, overlap parameters, a current modulation profile, and so on. A method for calculating the weighting function for the selected temporal window width is described further herein with regard to FIG. 5.

At 330, method 300 may include generating a final reconstructed image volume using the weighting function and Fourier blending. For example, the basis images may be Fourier blended in accordance with the weighting function to generate a final reconstructed image volume with the specified temporal window. For example, the basis images may be Fourier transformed to the Fourier, or frequency, domain using, for example, a fast Fourier transform (FFT) algorithm. In one example, the frequency data of each basis image may be multiplied by the weighting function. In some examples, the frequency data may be filtered, for example Fourier components with a frequency above a threshold frequency may be removed or adjusted, in order to smooth irregularities and noise in the data. After masking the basis image frequency data according to the weighting function, each masked frequency representation may be transformed back to the spatial (i.e., image) domain via an inverse Fourier transform (IFT). The inverse transformed images may then be summed to generate a final reconstructed image volume with the selected temporal width.

In some examples, the masked basis image frequency data may be summed in the frequency domain prior to applying the IFT. In such examples, the final reconstructed image volume is the result of the IFT.

In some embodiments, the Fourier transform process may be performed voxel by voxel to account for differences between voxels. However, in other embodiments, the process may be performed slice by slice, sub-region by sub-region so a number of multi-dimensional FFTs and/or multi-dimensional IFTs may be performed.

Continuing at 335, method 300 may include outputting the final reconstructed image volume. For example, the final reconstructed image volume may be output to a display 232 for review by an operator or a physician. As another example, the final reconstructed image volume may be output, for example, to mass storage 218 for reviewing at a later time. As yet another example, the final image volume may be saved to mass storage 218 and displayed on display 232.

At 340, method 300 may include determining if a new temporal window width selection is received. A new temporal window width selection may be received if a user selects a different temporal window width using, for example, the temporal window input 221. If a new temporal window width selection is not received, then no further processing is necessary and method 300 may end.

However, if a new temporal window width selection is received, method 300 may return to 325. At 325, method 300 may include calculating a weighting function for the new selected temporal window width, which may then be used at 330 to generate a new final reconstructed image volume with the new selected temporal window width using Fourier blending as described hereinabove. The new image volume may be output to a display 232 and/or mass storage 218 before method 300 continues again to 340. Method 300 may continue generating new image volumes with a specified temporal window in this manner until no new temporal window width selection is received at 340, at which point method 300 may end.

In this way, real-time, wide-cone image reconstructions with arbitrary temporal windows are described and may incorporate algorithms or approaches discussed herein. In some embodiments, initial reconstruction volumes are saved and combined using appropriately weighted summations in the Fourier domain to approximate analytic reconstruction of each phase. This allows a reviewer to scroll through the phases interactively and to select the optimal portion in the cardiac cycle to visualize each portion of the anatomy. In one implementation the need for back-projection may be eliminated for the real-time reconstruction, allowing the approach to take advantage of FFT techniques.

FIG. 4 is a high-level block diagram 400 illustrating an example method of reconstructing an image volume with a user-specified temporal window from two half-scan reconstructed image volumes according to an embodiment of the invention. In particular, diagram 400 illustrates the method 300 described hereinabove for reconstructing a blended image volume based on two basis images.

Calibrated projection data 405 may be used to reconstruct 407 two half-scan reconstructed image volumes 410 and 412, each with a same temporal window 415. Furthermore, the image volumes 410 and 412 may have different centerviews and may overlap as depicted in FIG. 4 and as described hereinabove with regard to FIG. 3.

The half-scan image volumes 410 and 412, or basis images, may both comprise inputs 420 for Fourier blending 425. Temporal window (TW) selection 427 may also comprise an input 428 for Fourier blending 425. Fourier blending 425 may comprise combining the basis images 410 and 412 in the Fourier domain according to a weighting function, where the weighting function is determined by the temporal window 427. The output 430 of Fourier blending 425 may comprise a blended reconstruction 435.

In one example, the blended reconstruction 435 may have a centerview corresponding to a view directly between the centerviews of basis images 410 and 412. The centerview of blended reconstruction may be aligned, as a non-limiting illustrative example, with a cardiac phase of interest. For this reason, the basis images 410 and 412 may in fact be generated such that the view halfway between the centerviews of the basis images 410 and 412 corresponds to the desired cardiac phase to be imaged. It should be appreciated that in some examples, the centerview of blended reconstruction 435 may be located at an arbitrary view, thereby allowing an operator to select, for example, a cardiac phase of interest even if the cardiac phase is not initially aligned with the central angle between the centerviews of basis images 410 and 412.

The blended reconstruction 435 may be reconstructed with a selected temporal window (TW) of the temporal windows 440. In particular, a blended reconstruction 435 with a temporal window TW₁ may comprise a half-scan reconstruction, where the blended reconstruction 435 includes data acquired over 180 degrees of the gantry rotation. The half-scan reconstruction offers the smallest temporal window possible while still being able to completely image the volume. Meanwhile, the temporal window TW₅ may comprise a full-scan reconstruction, where the blended reconstruction 425 includes data acquired over the entire 360 degrees of the gantry rotation. The temporal windows TW₂, TW₃, and TW₄ represent intermediate temporal windows between the temporal window extrema TW₁ and TW₅. It should be appreciated that the five temporal windows 440 depicted are for illustrative purposes, and that in some embodiments more or fewer temporal windows are possible.

FIG. 5 is a high-level flow chart illustrating an example method for generating a weighting function according to an embodiment of the invention. Method 500 may comprise a subroutine of method 300. Specifically, method 500 may comprise step 325 of method 300. Method 500 may be described with regard to the system and components depicted in FIGS. 1 and 2, however the method may be applied to other systems and components without departing from the scope of the present disclosure. Furthermore, method 500 may be described with reference to the set of graphs 600 shown in FIG. 6, where the set of graphs 600 illustrate an example construction of a weighting function according to method 500. The horizontal coordinate theta of each of the set of graphs 600 corresponds to view angle, with zero defined as the centerview of the final image.

The reconstruction weights for a weighting function should sum to one for conjugate samples (i.e., view data separated by 180 degrees). Therefore, an ideal weighting function according to noise performance would be piece-wise constant, with equal weights for conjugate samples. However, sharp discontinuities in reconstruction weights can cause streaking artifacts. To avoid such artifacts, the weighting function should be piece-wise continuous. The following constraints may be used in the computation of a weighting function: the weighting of conjugate samples should sum to one, the weighting function should be piece-wise continuous, and the weighting function should be equal to zero outside of the range of data being used. The algorithm for defining the weighting function is a two-step process. First, an outer transition region similar to the smooth transition from zero to one in the half-scan weighting function is defined. Then, the inner center region is defined using the outer region and normalized to guarantee that conjugate samples sum to one.

Method 500 may begin at 505. At 505, method 500 may include receiving a temporal window width selection. As an example, the temporal window width selection may be received from the temporal window input 221. In this way, the temporal window width may be selected by an operator.

At 510, method 500 may include calculating a boundary between an outer transition region and a central region. The boundary B, or boundary view angle, may be computed as

$$B = max [ \frac{{λ}_{trans}}{floor ( \frac{TW}{180} )} , TW - floor ( \frac{TW}{180} ) ] ,$$

where TW comprises the selected temporal window width and λₜᵣₐₙₛ comprises the overlap transition parameter described hereinabove.

At 515, method 500 may include defining an outer transition region with a specified slope. As an example, graph 610 includes a plot 615 of an outer transition region t(θ). The centerview of the final image volume may correspond to angle theta equal to zero. An outer transition region t(θ) may comprise regions between a first view θᵢ to the boundary B and between the boundary B and a last view θ_{f}. Specifically, as shown by plot 615, t(θ) may be defined with a slope for views θ in the ranges [−θ_{f}, −B) and (B, θ_{f}], and zero otherwise.

At 520, method 500 may include defining an intermediate central function based on the outer transition region. For example, the intermediate central function ƒ(θ) may be defined on the range [−B, B], and may be computed as the outer transition region multiplied by a discrete probability distribution within this range. Graph 620 includes a plot 625 of an example intermediate central function.

At 525, method 500 may include calculating a number of conjugate samples within the central region. Conjugate samples may comprise view data that is 180 degrees apart. In other words, conjugate samples comprise data acquired along the same line of sight but in opposite directions. Graph 630 includes a plot 635 of a number of conjugate samples within the central region. As shown by plot 635, there may be, for example, one conjugate sample close to the centerview and two conjugate samples on either side of the centerview.

At 530, method 500 may include defining a central region based on the intermediate central function. For example, given an intermediate central function ƒ(θ), then the central region may be defined as C(θ)=1−ƒ(θ) on the range [−B, B].

Continuing at 535, method 500 may include normalizing the central region using the calculated number of conjugate samples. To normalize the central region using the number of conjugate samples N_{w}(θ), the central region C(θ) may be divided by N_{w}(θ). Graph 640 includes a plot 645 of the normalized central region.

At 540, method 500 may include generating a weighting function based on the union of the outer transition region and the central region. That is, a weighting function W(θ) may be defined as W(θ)=t(θ)∪C(θ). Graph 650 includes a plot 655 of the weighting function based on the union of the outer transition region and the central region.

At 545, method 500 may include determining if a current (milliamp, or mA) modulation was applied during the scan. During scanning, current supplied to the x-ray source 104 may be modulated, for example, to compensate for greater attenuation through larger sections of anatomy, to save dose when imaging certain phases of the cardiac cycle, and so on. When this is the case, the reconstruction weights should reflect the current modulation. To that end, given a current profile, a new weighting function may be computed, for example, by multiplying the weighting function with the modulation profile and normalizing.

If current modulation was not applied during the scan, method 500 may continue to 550. At 550, method 500 may include outputting the weighting function W(θ) shown by plot 655 of graph 650. As described hereinabove with regard to FIG. 3, the weighting function may be used to generate a CT image with the temporal window built in to the weighting function. Method 500 may then end.

Returning to 545, if current modulation was applied during the scan, method 500 may continue to 555. At 555, method 500 may include retrieving the modulation profile. Graph 660 includes a plot 665 of an example current modulation profile m(θ). The modulation profile m(θ) shown by plot 665 is maximum about the centerview where theta equals zero, and decreases to a minimum current symmetrically about the centerview.

At 560, method 500 may include generating a modulation-compensated weighting function based on the weighting function and the modulation profile. To compensate for current modulation, the weighting function W(θ) may be multiplied by the modulation profile m(θ), so that a new weighting function W′(θ) may be defined as W′(θ)=W(θ)m(θ). Continuing at 565, method 500 may include normalizing the modulation-compensated weighting function. Normalization may comprise dividing the new weighting function W′(θ) by a normalization factor. Graph 670 includes a plot 675 of the normalized modulation-compensated weighting function.

At 570, method 500 may include outputting the normalized modulation-compensated weighting function. The normalized modulation-compensated weighting function W′(θ) may be used to generate a CT image according to the method described hereinabove with regard to FIG. 3. Method 500 may then end.

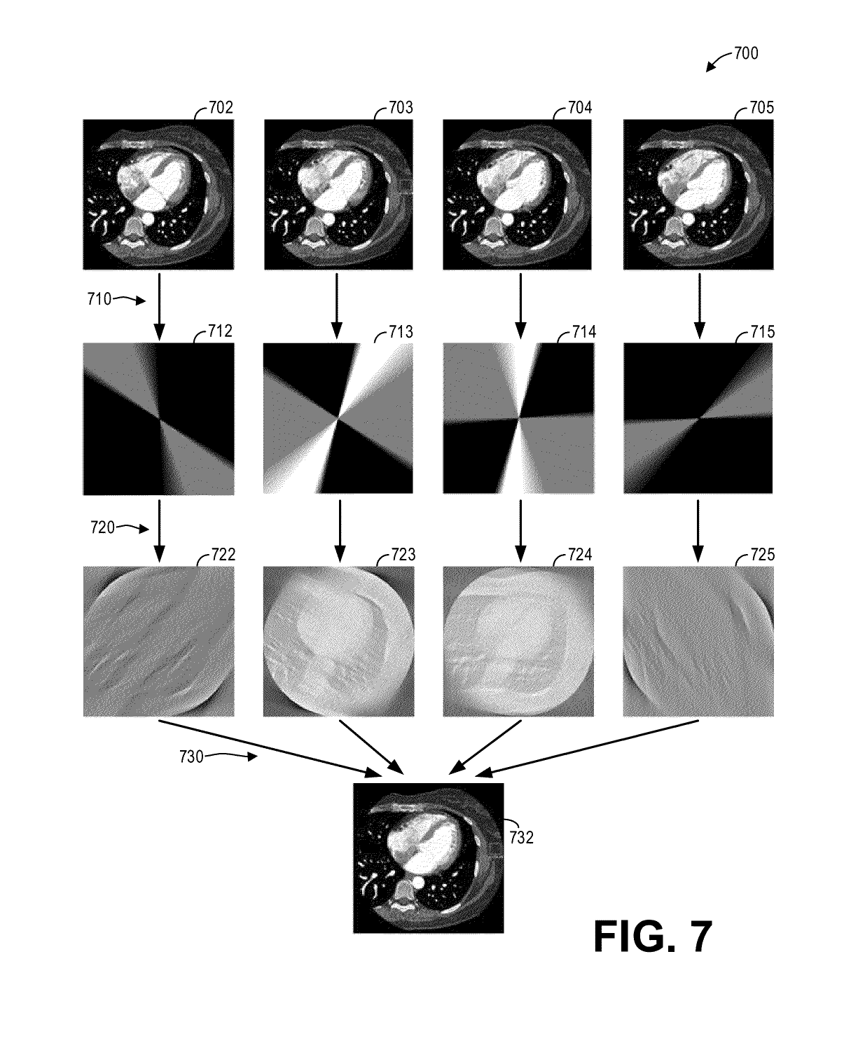

FIG. 7 shows a pictorial overview 700 of the generation of an image volume from basis images according to an embodiment of the invention. Overview 700 includes four basis images 702, 703, 704, and 705, where each basis image may comprise a different centerview. The basis images may comprise half-scan reconstructions, or may be reconstructed from data acquired over more than 180 degrees of views.

The basis images are Fourier transformed 710 into the frequency domain, for example using a FFT algorithm, and multiplied by a Fourier mask to produce the corresponding masked frequency images 712, 713, 714, and 715. The Fourier masks applied to each basis image in frequency space comprise two-dimensional polar representations of the one-dimensional weighting function described herein above with regard to FIG. 5, and the masks may be uniform in the radial direction. In this way, the Fourier masks may appropriately weight the conjugate samples in each basis image and may furthermore establish a selected temporal width. The masked frequency images may then be inverse Fourier transformed 720 into the spatial (i.e., image) domain to produce the corresponding masked basis images 722, 723, 724, and 725. The masked basis images 722, 723, 724, and 725 may then be summed 730 to produce the final image volume 732 with the selected temporal width.

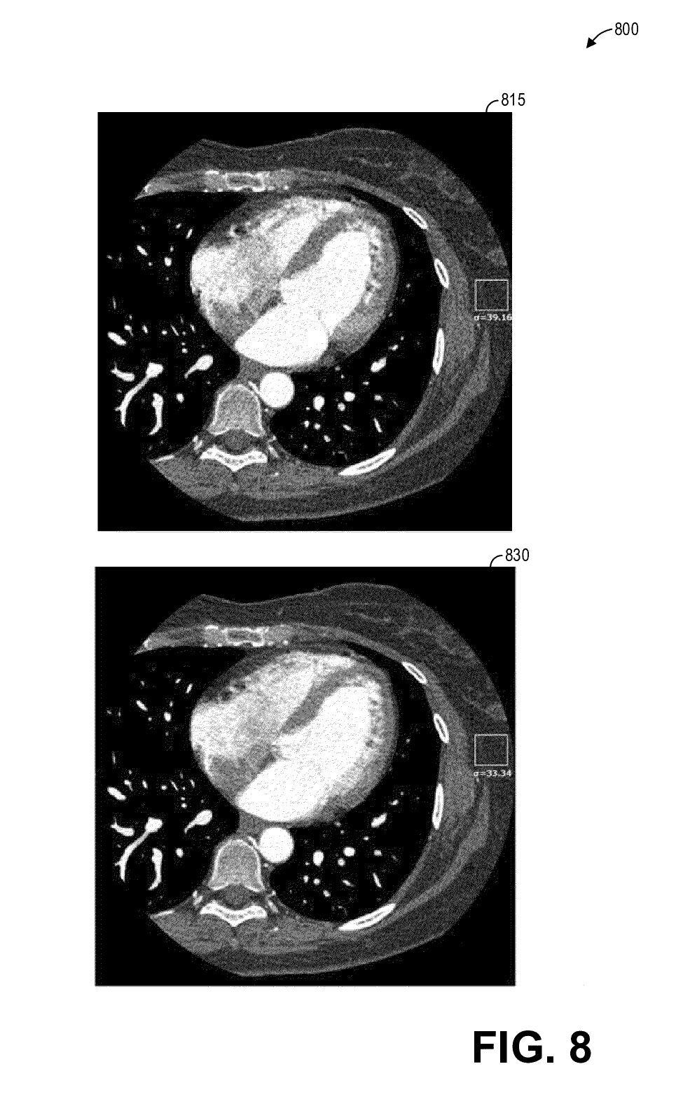

FIG. 8 shows a set of example image volumes 800 with different temporal windows according to an embodiment of the invention. In particular, the image volumes 800 include an original half-scan basis image volume 815 and a temporally thickened image volume 830 (i.e., an image volume with a temporal width greater than a half-scan reconstruction) generated using the approach described herein. The standard deviation of noise σ for the temporally thickened image volume 830 may be lower than the standard deviation of noise σ for the original image, however as shown there may also be a reduction in edge resolution in the temporally thickened image volume 830. According to the approach described herein, an operator may select a temporal width for a displayed image volume to find a balance between noise level and resolution.

A technical effect of the disclosure may include the real-time generation of a diagnostic image with an arbitrary temporal width using basis images reconstructed from acquired x-ray projection data. Another technical effect of the disclosure may include the calculation of a weighting function according to various conditions for use in generating the diagnostic image using the basis images.

In one embodiment, a method for imaging comprises acquiring x-ray projection data; calibrating the x-ray projection data; generating at least two basis image volumes by reconstructing the calibrated x-ray projection data, each of the at least two basis image volumes including different centerviews and a temporal width corresponding to an x-ray source rotation between 180 degrees and 360 degrees; and generating a final image volume by Fourier blending the at least two basis image volumes based on a selected temporal window width. Fourier blending the at least two basis image volumes comprises transforming the at least two basis image volumes into a frequency domain, combining the at least two basis image volumes in the frequency domain, and transforming the combination into a spatial domain. In one example, the selected temporal window width is selected by a user from among a limited range. The at least two basis image volumes include different centerviews, and wherein a centerview of the final image volume is located between the different centerviews. In one example, the at least two basis image volumes overlap by a specified non-zero amount.

In one example, the selected temporal window width is selected by a user-provided input, and Fourier blending the at least two basis image volumes comprises weighting the at least two basis image volumes based on the user-provided temporal window width. In another example, the selected temporal window is automatically selected to optimize one or more image parameters. In another example, weighting the at least two basis image volumes is further based on an overlap of the at least two basis image volumes. In yet another example, weighting the at least two basis image volumes is further based on a current modulation applied to an x-ray source. In another example, weighting the at least two basis image volumes is further based on a normalization condition and a piece-wise continuity condition.

In another embodiment, a method for medical imaging comprises generating a first diagnostic image with a first temporal window by blending basis images, displaying the first diagnostic image, generating a second diagnostic image with a second temporal window by blending the basis images responsive to receiving a selection of the second temporal window, and displaying the second diagnostic image. In one example, the selection is performed by a user.

In one example, the basis images comprise at least two half-scan image volumes reconstructed from calibrated x-ray projection data. In one example, the at least two half-scan image volumes are reconstructed using an analytic reconstruction approach. In another example, the at least two half-scan image volumes are reconstructed using an iterative reconstruction approach. In some examples, the calibrated x-ray projection data is acquired over at least a full rotation.

In one example, the method further comprises saving the first diagnostic image in non-transitory memory and displaying the first diagnostic image retrieved from the non-transitory memory responsive to receiving a selection of the first temporal window.

In yet another embodiment, a system for medical imaging comprises an operator console including a user input for a temporal window selection, and a display device. The system further comprises a processor operably connected to the operator console and the display device, and configured with executable instructions in non-transitory memory that when executed cause the processor to generate an image volume comprising a combination of at least two half-scan volumes based on the temporal window selection received from the operator console, and output the image volume to the display device for display. For example, generating the image volume comprises Fourier blending the at least two half-scan volumes with a weighting function computed using the temporal window selection.

In one example, the processor is further configured with executable instructions in the non-transitory memory that when executed cause the processor to generate a second image volume comprising a combination of the at least two half-scan volumes responsive to receiving a second temporal window selection from the operator console and output the second image volume to the display device for display.

In another example, the processor is further configured with executable instructions in the non-transitory memory that when executed cause the processor to generate the at least two half-scan volumes based on calibrated x-ray projection data.

In yet another embodiment, a method for medical imaging comprises generating a diagnostic image by blending at least two half-scan image volumes reconstructed from calibrated x-ray projection data based on a selected temporal window width.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for imaging, comprising:
acquiring x-ray projection data;
calibrating the x-ray projection data;
generating at least two basis image volumes by reconstructing the calibrated x-ray projection data, each of the at least two basis image volumes including different centerviews and a temporal width corresponding to an x-ray source rotation between 180 degrees and 360 degrees; and
generating a final image volume by Fourier blending the at least two basis image volumes based on a selected temporal window width.

2. The method of claim 1, wherein Fourier blending the at least two basis image volumes comprises:
transforming the at least two basis image volumes into a frequency domain;
combining the at least two basis image volumes in the frequency domain; and
transforming the combination into a spatial domain.

3. The method of claim 1, wherein the selected temporal window width is selected by a user-provided input, and wherein Fourier blending the at least two basis image volumes comprises weighting the at least two basis image volumes based on the user-provided temporal window width.

4. The method of claim 3, wherein weighting the at least two basis image volumes is further based on an overlap of the at least two basis image volumes.

5. The method of claim 3, wherein weighting the at least two basis image volumes is further based on a current modulation applied to an x-ray source.

6. The method of claim 3, wherein weighting the at least two basis image volumes is further based on a normalization condition and a piece-wise continuity condition.

7. The method of claim 1, wherein the selected temporal window is automatically selected to optimize one or more image parameters.

8. The method of claim 1, wherein the selected temporal window width is selected by a user from among a limited range.

9. The method of claim 1, wherein a centerview of the final image volume is located between the different centerviews.

10. The method of claim 1, wherein the at least two basis image volumes overlap by a specified non-zero amount.

11. A method for medical imaging, comprising:
generating a first diagnostic image with a first temporal window by blending basis images;
displaying the first diagnostic image;
generating a second diagnostic image with a second temporal window by blending the basis images responsive to receiving a selection of the second temporal window; and
displaying the second diagnostic image.

12. The method of claim 10, wherein the basis images comprise at least two half-scan image volumes reconstructed from calibrated x-ray projection data.

13. The method of claim 11, wherein the at least two half-scan image volumes are reconstructed using an analytic reconstruction approach.

14. The method of claim 11, wherein the calibrated x-ray projection data is acquired over at least a full rotation.

15. The method of claim 10, further comprising saving the first diagnostic image in non-transitory memory and displaying the first diagnostic image retrieved from the non-transitory memory responsive to receiving a selection of the first temporal window.

16. The method of claim 10, wherein the selection is performed by a user.

17. A system for medical imaging, comprising:
an operator console including a user input for a temporal window selection;
a display device; and
a processor operably connected to the operator console and the display device, the processor configured with executable instructions in non-transitory memory that when executed cause the processor to:
generate an image volume comprising a combination of at least two basis image volumes based on the temporal window selection received from the operator console; and
output the image volume to the display device for display.

18. The system of claim 17, wherein the processor is further configured with executable instructions in the non-transitory memory that when executed cause the processor to generate a second image volume comprising a combination of the at least two basis image volumes responsive to receiving a second temporal window selection from the operator console and output the second image volume to the display device for display.

19. The system of claim 17, wherein the processor is further configured with executable instructions in the non-transitory memory that when executed cause the processor to generate the at least two basis image volumes based on calibrated x-ray projection data.

20. The system of claim 17, wherein generating the image volume comprises Fourier blending the at least two basis image volumes with a weighting function computed using the temporal window selection.

21. A method for medical imaging, comprising:
generating a diagnostic image by blending at least two half-scan image volumes reconstructed from calibrated x-ray projection data based on a selected temporal window width.
